(19) United States
(12) Patent Application Publication
(10) Pub. No.: US 2009/0238898 A1
(43) Pub. Date: Sep. 24, 2009

(54) METHOD OF INHIBITING THE PROLIFERATION AND MIGRATION OF HELICOBACTOR PYLORI

(76) Inventors: Hiroaki Takeuchi, Kochi (JP); Yoshinari Ikegami, Hyogo (JP); Takeshi Yasukawa, Hyogo (JP); Koji Nakagawa, Hyogo (JP)

Correspondence Address: KNOBBE MARTENS OLSON & BEAR LLP, 2040 MAIN STREET, FOURTEENTH FLOOR, IRVINE, CA, 92614, US

(21) Appl. No.: 12/067,918

(22) PCT Filed: Sep. 25, 2006
(86) PCT No.: PCT/JP2006/318983

§ 371 Date: Apr. 15, 2008

(30) Foreign Application Priority Data
Sep. 26, 2005 (JP) 2005-278540

(51) Int. Cl. A61K 33/06; A61K 33/00

(52) U.S. Cl. 424/682; 424/722

(57) ABSTRACT

Helicobacter pylori strains are indigenous to each human host and infect and colonize in the stomach of the host, and they are also known to be involved in diseases of the upper gastrointestinal tracts, among others. The present invention provides an economical method for inhibiting the proliferation and migration of Helicobacter pylori strains without being concerned about emergence of resistant bacteria or side effects due to drugs traditionally used to remove Helicobacter pylori strains, as well as a beverage or food utilizing such method, by selecting from among mineral-rich waters obtained from deep sea water, and by ingesting, a water or waters capable of inhibiting the proliferation and migration of Helicobacter pylori strains that are indigenous to the host and infecting and colonized in the stomach of the host.

### TECHNICAL FIELD

The present invention relates to a method for inhibiting the proliferation and migration of Helicobacter pylori strains that are considered a cause of gastric ulcer, duodenal ulcer, stomach cancer, etc.

More specifically, the present invention relates to a method for inhibiting the proliferation and migration of Helicobacter pylori strains in a manner not producing resistant bacteria to, or harmful side effects from treatment drugs of, Helicobacter pylori strains that are indigenous to the host.

For clarification purposes, in the context of the present invention a method for inhibiting the proliferation and migration of Helicobacter pylori strains refers to both a method for inhibiting the proliferation of Helicobacter pylori strains and a method for inhibiting the active migration of Helicobacter pylori strains.

Pumping of deep sea water began in Japan in 1989 off the coast of Cape Muroto in Kochi Prefecture using a deep sea water collection facility constructed on land. Today, deep sea water is collected in various parts of Japan including Toyama Prefecture and Okinawa Prefecture. It is well-known that deep sea water has various beneficial characteristics such as containing a lot of nutrients, being clean, and remaining stable at low temperature. Accordingly, many products that use deep sea water have been developed in recent years. Among others, there is a trend of active utilization of deep sea water in the food industry in the production of drinking water, processed seafood products, fermented products, etc., and a number of products using deep sea water are now available on the market. Many of these products are drawing the attention as offering the benefits of deep sea water such as cleanliness and high mineral content, and utilized as a pure material used in product manufacturing or a source of quality minerals.

On the other hand, medical and scientific studies are gradually finding the functions and mechanisms embodied in the human body by drinking water that uses deep sea water and thereby contains a lot of minerals. Although deep sea water is expected to offer numerous benefits, such as regulating the actions of the intestines, having positive effects on the immune strength of the body, and preventing lifestyle-related diseases, the specific actions and effects of deep sea water including those mentioned above are not yet understood fully.

On the other hand, Helicobacter pylori strains infect and colonize in the human stomach and are reported to have a hand in the development of diseases of the upper gastrointestinal tracts as well as autoimmune diseases, acute coronary diseases, and lifestyle-related diseases, among others. In particular, it is a well-known fact that Helicobacter pylori strains are closely related to the causes of gastric ulcer, duodenal ulcer, stomach cancer, etc.

For this reason, various therapies are used to remove Helicobacter pylori strains, such as those using various antibacterial agents, proton pump inhibitors (PPIs) that inhibit secretion of gastric acid, and antibiotics. As a specific example, a local oral agent that contains a bismuth compound, such as bismuth citrate, to kill Helicobacter pylori strains in the oral cavity is disclosed in Patent Literature 1 (Japanese Patent Laid-open No. Hei 8-20543). Also, Patent Literature 2 (Japanese Patent Laid-open No. Hei 8-48629) and Patent Literature 3 (Japanese Patent Laid-open No. Hei 9-208578) disclose anti-pylori agents exhibiting excellent antibacterial activity against Helicobacter pylori strains, where the effective ingredient is pyridone carboxylic acid, its ester, or salt thereof.

In addition to the above, Patent Literature 4 (Japanese Patent Laid-open No. Hei 9-295938) discloses an ulcer treatment drug, whose effective ingredients include a carbapenem compound or its pharmacologically acceptable form of salt having strong antibacterial activity against Helicobacter pylori strains that colonize and grow in the internal membranes of the stomach, for use as a drug to treat digestive ulcers and chronic superficial gastritis as well as prevent their recurrence.

In addition, Patent Literature 5 (Japanese Patent Laid-open No. 2000-63280) and Patent Literature 6 (Japanese Patent Laid-open No. Hei 10-109942) also disclose drugs that combine coptis root, cork tree bark or other galenical preparation in powder or extract form having antibacterial activity against Helicobacter pylori strains, as an effective medicinal composition to treat or prevent gastrointestinal diseases by effectively inhibiting the proliferation of, or removing and discharging, Helicobacter pylori strains that are considered a cause of the so-called digestive ulcers such as gastritis, gastric ulcer and duodenal ulcer, with at least one ingredient selected from histamine H₂ receptor antagonists, proton pump inhibitors, gastric-mucosa protective drugs for gastritis and digestive ulcers, gastric anti-acid drugs, and anti-diarrheal drugs.

Furthermore, Patent Literature 7 (Japanese Patent Laid-open No. Hei 11-180888) discloses a polyphenol obtained from fruits of rosaceous plants as an effective ingredient for use in drugs, anti-infection agents, or food, having an antibacterial action against Helicobacter pylori strains, while Patent Literature 8 (Japanese Patent Laid-open No. Hei 11-292788) discloses a Fe-bonded lactoferrin for use in anti-infection agents, drugs or beverages/food. Similarly, Patent Literature 9 (Japanese Patent Laid-open No. 2002-68992) discloses a beverage/food or food additive containing tea polyphenol as an effective ingredient along with a proton inhibitor, while Patent Literature 10 (Japanese Patent Laid-open No. 2005-68014) discloses an antibacterial agent against Helicobacter pylori strains, constituted by one or two or more extracts selected from the group that includes Apocynum venetum L., Vaccinium myrtillus L. and Acanthopanax senticosus Harms.

However, the types of Helicobacter pylori strains present in the body are different from one host to another, and accordingly the effects of general antibacterial agents are not clear and cases of unsuccessful removal of Helicobacter pylori due to emergence of resistant bacteria and side effects are increasing.

For the aforementioned reasons, a number of simple methods for detecting and identifying Helicobacter pylori strains have been reported, including: an infection diagnosis probe useful in the detection and identification of Helicobacter pylori strains themselves, as disclosed in Patent Literature 11 (Japanese Patent Laid-open No. Hei 10-33179); a real-time infection inspection method based on the principles of “analysis of expired gas using ¹³C labeled urea,” as disclosed in Patent Literature 12 (Japanese Patent Laid-open No. Hei 10-87512; a primer based on Helicobacter pylori strain genes and a method for examining clarithromycin resistance of Helicobacter pylori strains, as disclosed in Patent Literature 13 (Japanese Patent Laid-open No. Hei 10-285099) and Patent Literature 14 (Japanese Patent Laid-open No. 2001-321197); and a detection method using an enzyme urease derived from Helicobacter pylori strains, as disclosed in Patent Literature 15 (Japanese Patent Laid-open No. Hei 11-318490). However, utilization of deep sea water in the inhibition of proliferation or migration of Helicobacter pylori strains has not been reported to date.

Patent Literature 1: Japanese Patent Laid-open No. Hei 8-20543
Patent Literature 2: Japanese Patent Laid-open No. Hei 8-48629
Patent Literature 3: Japanese Patent Laid-open No. Hei 9-208578
Patent Literature 4: Japanese Patent Laid-open No. Hei 9-295938

### Patent Literature 5: Japanese Patent Laid-open No. 2000-63280

Patent Literature 6: Japanese Patent Laid-open No. Hei 10-109942
Patent Literature 7: Japanese Patent Laid-open No. Hei 11-180888
Patent Literature 8: Japanese Patent Laid-open No. Hei 11-292788

### Patent Literature 9: Japanese Patent Laid-open No. 2002-68992

### Patent Literature 10: Japanese Patent Laid-open No. 2005-68014

Patent Literature 11: Japanese Patent Laid-open No. Hei 10-33179
Patent Literature 12: Japanese Patent Laid-open No. Hei 10-87512
Patent Literature 13: Japanese Patent Laid-open No. Hei 10-285099

### Patent Literature 14: Japanese Patent Laid-open No. 2001-321197

Patent Literature 15: Japanese Patent Laid-open No. Hei 11-318490

### SUMMARY OF THE INVENTION

### Problems to Be Solved by the Invention

As mentioned above, methods for preventing or treating gastrointestinal diseases are being sought, which are able to accommodate the diversity of Helicobacter pylori strains closely related to the causes of gastric ulcer, duodenal ulcer, stomach cancer, etc., and thereby suppress side effects of antibacterial agents and emergence of resistant bacteria. In the meantime, inhibition of proliferation and migration of Helicobacter pylori strains itself provides a means for prevention and treatment of diseases caused by Helicobacter pylori strains. When prevention is considered, it is safer for the health of human body and also provides an easier method of intake to continuously ingest as part of meals a food matter having the effect of inhibiting the proliferation and migration of Helicobacter pylori strains, instead of using a drug regularly.

In light of the above, it is an object of the present invention to achieve inhibition of proliferation and migration of Helicobacter pylori strains through utilization of inexpensive materials, such as drinking water and other substances we consume daily, because such is an ideal way to inhibit the proliferation and migration of Helicobacter pylori strains.

It is another object of the present invention to establish a technology for inhibiting the proliferation and migration of Helicobacter pylori strains that colonize in and infect the human stomach and are also reported to have a hand in diseases of the upper gastrointestinal tracts as well as autoimmune diseases, acute coronary diseases, and lifestyle-related diseases, among others, by focusing on deep sea water, which is rich in nutrients and minerals and inexpensive and therefore used widely in the food industry for use in the production of drinking water, processed seafood products, fermented products, etc., because deep sea water can be consumed easily in everyday life as drinking water.

### Means for Solving the Problems

In light of the above, the present invention establishes a technology for inhibiting the proliferation and migration of Helicobacter pylori strains by utilizing drinking water and other substances consumed daily, based on a discovery of a method for inhibiting the proliferation and migration of Helicobacter pylori strains through substances containing minerals. To be specific, the present invention represents a newly discovered method for inhibiting the proliferation and migration of Helicobacter pylori strains by utilizing a liquid that contains at least one or more of magnesium, calcium, sodium and potassium, based on the results of examination with focus on magnesium, calcium, sodium and potassium that exist in particularly high quantities in deep sea water among the various minerals contained in deep sea water, and also with focus on liquids that can be used as a way to ingest these minerals easily in everyday life, and consequently the present invention establishes a technology for inhibiting the proliferation and migration of Helicobacter pylori strains by utilizing drinking water and other substances consumed daily. The minerals examined in connection with the present invention are derived from deep sea water subject to minimal contamination by harmful substances, etc., and thus can be used favorably as materials for beverages and food. However, it is not necessary to limit the source of these minerals to deep sea water. If the mineral content per kilogram is 40 mg or more, proliferation and migration of Helicobacter pylori strains can be inhibited.

If the mineral content per kilogram is 5,900 mg or more, proliferation and migration of all Helicobacter pylori strains can be inhibited. From the viewpoint of cost effectiveness, there is no need to increase the mineral content to beyond 5,900 mg per kilogram.

### EFFECTS OF THE INVENTION

The greatest benefit of the present invention is that by consuming mineral-rich water obtained from deep sea water that is conveniently available, proliferation and migration of Helicobacter pylori strains indigenous to the host can be inhibited to prevent and treat diseases of the upper gastrointestinal tracts, among others, without being concerned about emergence of resistant bacteria and side effects of treatment drugs.

### BRIEF DESCRIPTION OF THE DRAWINGS

FIG. 1. Condition of colonies that grew in an agar culture medium

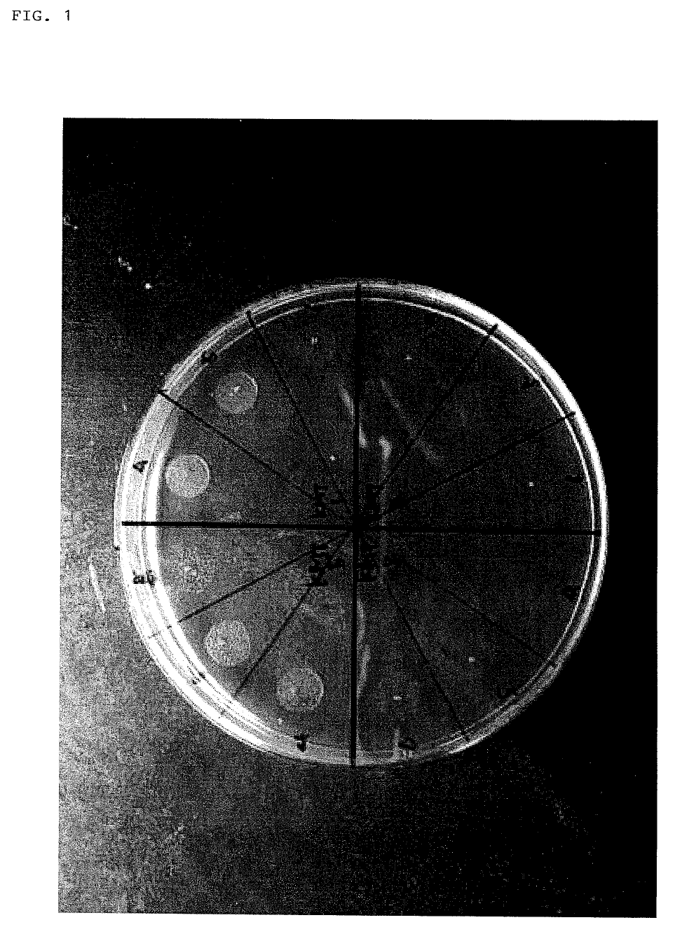

### BEST MODE FOR CARRYING OUT THE INVENTION

### [Method for Testing Inhibition of Proliferation of Helicobacter Pylori Strains]

The method for testing the inhibition of proliferation of Helicobacter pylori strains used in the present invention is explained below.

Each type of Helicobacter pylori strains corresponding to approx. 1 platinum loop was taken from an agar culture medium (Table 1) on the second day of culture and suspended in 1 ml of 2.8% Brucella broth culture solution so that O.D.₆₀₀ became 0.3.

The prepared solution was then diluted to 10⁻⁴, 10⁻⁵ and 10⁻⁶ times, respectively, using sterilized water, and each bacteria-suspended solution was dripped by 10 μl each into agar culture mediums (Table 3) containing mineral-rich waters A, B, C, D, E, F and G (test waters) prepared from deep sea water as shown in Table 2. Next, the culture mediums were cultured for three days in an environment of 37° C. and 10% CO₂, after which the CFU (colony forming unit=viable bacterial cell count) of each culture medium was measured.

For your reference, Milli-Q water was used as a control water.

TABLE 1
Composition of agar culture medium

| Composition | Content | |
|---|---|---|
| Brucella Broth | 2.8 | g |
| Milli-Q water | 90 | ml |
| Agar powder | 1.4 | g |
| Horse serum | 10 | ml |
| Vancomycin solution (100 mg/ml) | 10 | μl |

TABLE 2
Compositions of mineral-rich waters (mg/kg)

| | |
|---|---|
| Mineral-rich | Ion type |

| water | Mg | Ca | Na | K |
|---|---|---|---|---|
| A | 100 | 200 | 50 | 50 |
| B | 150 | 150 | 50 | 50 |
| C | 200 | 70 | 65 | 65 |
| D | 400 | — | — | — |
| E | — | 400 | — | — |
| F | — | — | 400 | — |
| G | — | — | — | 400 |

TABLE 3
Composition of agar culture medium

| Composition | Content | |
|---|---|---|
| Brucella Broth | 2.8 | g |
| Test water or control water | 90 | ml |
| Agar powder | 1.4 | g |
| Horse serum | 10 | ml |
| Vancomycin solution (100 mg/ml) | 10 | μl |

### [Helicobacter Pylori Strains Used]

In the test, mainly clinically isolated strains were used.

KMT series: Clinically isolated strain obtained from the Medical School of Kochi University
NY series: Strain resistant to antibiotics (metronidazole and clarithromycin) (made in Japan)
26695: Clinically isolated strain analyzed in details to gene level (obtained from Europe)
NCTC11637: Clinically isolated strain (obtained from the U.S.)
HPK5: Clinically isolated strain (obtained in Japan)
HPKT510: Broken strain of cdrA gene (gene relating to cell division) of HPK5 prepared by means of gene recombination

### [Method for Evaluating Proliferation Inhibition]

The effects of inhibiting the proliferation of Helicobacter pylori strains in test waters A, B, C, D, E, F and G were determined based on the CFU (colony forming unit=viable bacterial cell count) of the control water being 100%.

By considering the variations resulting from the operations carried out in the test, test waters whose CFU was smaller than 80% of the CFU of the control water were considered effective.

The test was conducted three times and if effectiveness was found in two test runs, the test water was judged effective in inhibiting Helicobacter pylori proliferation (indicated by ◯).

### [Method for Testing Inhibition of Migration of Helicobacter Pylori Strains]

Using a sterilized toothpick, bacteria of each type were taken from agar culture mediums in which Helicobacter pylori strains had been cultured, and then transferred to Brucella broth agar culture mediums (Table 4) using mineral-rich waters A, B, C, D, E, F and G prepared from deep sea water and also using Milli-Q water as a control. Next, the culture mediums were cultured for three days in an environment of 37° C. and 10% CO₂, after which the diameters of colonies in each culture medium were measured.

TABLE 4
Composition of agar culture medium

| Composition | Content | |
|---|---|---|
| Brucella Broth | 2.8 | g |
| Test water or control water | 90 | ml |
| Agar powder | 0.35 | g |
| Horse serum | 10 | ml |
| Vancomycin solution (100 mg/ml) | 10 | μl |

### [Method for Evaluating Migration Inhibition]

In the evaluation, test waters whose colonies had a diameter smaller than the diameters of colonies in the control water were considered effective. The test was conducted three times and if effectiveness was found in two test runs, the test water was judged effective in inhibiting Helicobacter pylori migration (indicated by ⊙).

For your reference, colonies with a diameter less than 2 mm in the control were considered not sufficiently grown and not counted.

The present invention is explained below using examples with respect to test waters A, B, C, D, E, F and G. It should be noted, however, that mineral-rich waters prepared form deep sea water are not at all limited to these waters.

### Example 1

Table 5 shows the effects of test waters in inhibiting the proliferation of various types of Helicobacter pylori strains.

TABLE 5
Effects of inhibiting proliferation of various
types of Helicobacter pylori strains

| No. | Bacteria name | A | B | C | D | E | F | G |
|---|---|---|---|---|---|---|---|---|
| 1 | KMT27 | ◯ | ◯ | ◯ | ◯ | ◯ | | |
| 2 | KMT31 | | ◯ | | ◯ | ◯ | | |
| 3 | KMT40 | | ◯ | ◯ | | ◯ | ◯ | |
| 4 | KMT44 | | ◯ | | ◯ | | | ◯ |
| 5 | KMT45 | ◯ | ◯ | | ◯ | | ◯ | |
| 6 | KMT46 | | | ◯ | ◯ | ◯ | | |
| 7 | KMT47 | ◯ | ◯ | | ◯ | ◯ | | |
| 8 | KMT50 | ◯ | ◯ | ◯ | ◯ | ◯ | | |
| 9 | NY1 | | | | | | ◯ | ◯ |
| 10 | NY8 | | | ◯ | ◯ | | | |
| 11 | NY11 | | | | | | | |
| 12 | NY31 | | ◯ | ◯ | ◯ | ◯ | | |
| 13 | 26695 | | | | | | | |
| 14 | HPK5 | | | ◯ | ◯ | | ◯ | ◯ |
| 15 | HPKT510 | ◯ | | | | ◯ | | |
| 16 | 11637 | ◯ | ◯ | | ◯ | | | |

As shown in Table 5, whether proliferation of a given type of bacterial strain was inhibited or not varied depending on the type of test water.

Also, it is evident from Table 5 that test water D inhibited the proliferation of many types of bacterial strains, where clearly the effect of inhibiting Helicobacter pylori proliferation varies according to the type of bacterial strain.

Based on the above results, it is found that by consuming mineral-rich water conveniently available and obtained from deep sea water having the effect of inhibiting the proliferation of various types of Helicobacter pylori strains, diseases of the upper gastrointestinal tracts, among others, can be prevented and treated without being concerned about emergence of resistant bacteria and side effects of treatment drugs.

### Example 2

Table 6 shows the effects of inhibiting the migration of various types of Helicobacter pylori strains cultured in agar culture mediums using mineral-rich waters obtained from deep sea water.

TABLE 6
Effects of inhibiting migration of various
types of Helicobacter pylori strains

| No. | Bacteria name | A | B | C | D | E | F | G |
|---|---|---|---|---|---|---|---|---|
| 1 | KMT27 | | | | | | | |
| 2 | KMT31 | ⊙ | | | | ⊙ | | |
| 3 | KMT40 | | | | | | | |
| 4 | KMT44 | | ⊙ | ⊙ | ⊙ | ⊙ | | |
| 5 | KMT45 | | ⊙ | ⊙ | ⊙ | ⊙ | | |
| 6 | KMT46 | | ⊙ | ⊙ | ⊙ | ⊙ | | |
| 7 | KMT47 | | | | | | | |
| 8 | KMT50 | | | | | | | |
| 9 | NY1 | | | | | | | |
| 10 | NY8 | ⊙ | ⊙ | | | ⊙ | | |
| 11 | NY11 | ⊙ | ⊙ | | | ⊙ | | |
| 12 | NY31 | | | | | | | |
| 13 | 26695 | | | | | | | |
| 14 | HPK5 | | ⊙ | ⊙ | | ⊙ | | |
| 15 | HPKT510 | | | | | ⊙ | | |
| 16 | 11637 | | | | | | | |

As shown in Table 6, test waters B, C, D, and E are effective in inhibiting the migration of KMT44, 45 and 46 strains.

Based on the above results, it is found that by selecting proper mineral-rich waters, migration of Helicobacter pylori strains infecting and colonized in the host can be inhibited.

### Example 3

Starting from mineral-rich waters each having a total mineral content of 400 mg/kg, the total mineral content of each water was changed within a range of 20 to 11,800 mg/kg based on the percentages of ion contents shown in Table 7, and the effects of varying total mineral concentrations on the inhibition of proliferation of Helicobacter pylori strains were examined.

TABLE 7
Percentages of ion contents in various mineral waters (%)

| | |
|---|---|
| Mineral-rich | Ion type |

| water | Mg | Ca | Na | K |
|---|---|---|---|---|
| A | 25 | 50 | 12.5 | 12.5 |
| B | 37.5 | 37.5 | 12.5 | 12.5 |
| C | 50 | 17.5 | 16.25 | 16.25 |
| D | 100 | 0 | 0 | 0 |
| E | 0 | 100 | 0 | 0 |
| F | 0 | 0 | 100 | 0 |
| G | 0 | 0 | 0 | 100 |

By considering the variations resulting from the operations carried out in the test, test waters whose CFU was smaller than 80% of the CFU of the control water were considered effective. The test was conducted three times and if effectiveness was found in two test runs, the test water was judged effective in inhibiting Helicobacter pylori proliferation (indicated by $$◯$$).

For your reference, the strains used in the test were those Helicobacter pylori strains indicated in 1 through 16.

Tables 8 through 15 show the results of the examination where the total mineral concentration was gradually raised from 20 to 11,800 mg/kg.

TABLE 8
Total mineral concentration = 20 mg/kg

| No. | Bacteria name | A | B | C | D | E | F | G |
|---|---|---|---|---|---|---|---|---|
| 1 | KMT27 | | | | | | | |
| 2 | KMT31 | | | | | | | |
| 3 | KMT40 | | | | | | | |
| 4 | KMT44 | | | | | | | |
| 5 | KMT45 | | | | | | | |
| 6 | KMT46 | | | | | | | |
| 7 | KMT47 | | | | | | | |
| 8 | KMT50 | | | | | | | |
| 9 | NY1 | | | | | | | |
| 10 | NY8 | | | | | | | |
| 11 | NY11 | | | | | | | |
| 12 | NY31 | | | | | | | |
| 13 | 26695 | | | | | | | |
| 14 | HPK5 | | | | | | | |
| 15 | HPKT510 | | | | | | | |
| 16 | 11637 | | | | | | | |

TABLE 9
Total mineral concentration = 40 mg/kg

| No. | Bacteria name | A | B | C | D | E | F | G |
|---|---|---|---|---|---|---|---|---|
| 1 | KMT27 | | | ◯ | ◯ | | | |
| 2 | KMT31 | | | | | | | |
| 3 | KMT40 | | | | | | | |
| 4 | KMT44 | | | | | | | |
| 5 | KMT45 | | | | | | | |
| 6 | KMT46 | | | | | | | |
| 7 | KMT47 | | | | | | | |
| 8 | KMT50 | | | | | | | |
| 9 | NY1 | | | | | | | |
| 10 | NY8 | | | | | | | |
| 11 | NY11 | | | | | | | |
| 12 | NY31 | | ◯ | | ◯ | | | |
| 13 | 26695 | | | | | | | |
| 14 | HPK5 | | | | | | | |
| 15 | HPKT510 | | | | | | | |
| 16 | 11637 | | | | | | | |

TABLE 10
Total mineral concentration = 100 mg/kg

| No. | Bacteria name | A | B | C | D | E | F | G |
|---|---|---|---|---|---|---|---|---|
| 1 | KMT27 | | | ◯ | ◯ | | | |
| 2 | KMT31 | | | | | | | |
| 3 | KMT40 | | | | | | ◯ | |
| 4 | KMT44 | | | | | | | ◯ |
| 5 | KMT45 | | | | | | | |
| 6 | KMT46 | | | | | | | |
| 7 | KMT47 | | | | | | | |
| 8 | KMT50 | | ◯ | ◯ | | | | |
| 9 | NY1 | | | | | | | |
| 10 | NY8 | | | | | | | |
| 11 | NY11 | | | | | | | |
| 12 | NY31 | | ◯ | ◯ | ◯ | ◯ | | |
| 13 | 26695 | | | | | | | |
| 14 | HPK5 | | | | | | | |
| 15 | HPKT510 | | | | | | | |
| 16 | 11637 | | | | | | | |

TABLE 11
Total mineral concentration = 1,200 mg/kg

| No. | Bacteria name | A | B | C | D | E | F | G |
|---|---|---|---|---|---|---|---|---|
| 1 | KMT27 | ◯ | ◯ | ◯ | ◯ | ◯ | ◯ | ◯ |
| 2 | KMT31 | | ◯ | ◯ | ◯ | ◯ | | |
| 3 | KMT40 | | ◯ | ◯ | | ◯ | ◯ | |
| 4 | KMT44 | ◯ | ◯ | | ◯ | | ◯ | ◯ |
| 5 | KMT45 | ◯ | ◯ | ◯ | ◯ | ◯ | ◯ | ◯ |
| 6 | KMT46 | | | ◯ | ◯ | ◯ | | |
| 7 | KMT47 | ◯ | ◯ | ◯ | ◯ | ◯ | | |
| 8 | KMT50 | ◯ | ◯ | ◯ | ◯ | ◯ | | |
| 9 | NY1 | | | | | | ◯ | ◯ |
| 10 | NY8 | | | ◯ | ◯ | | | |
| 11 | NY11 | | | | | | | |
| 12 | NY31 | | ◯ | ◯ | ◯ | ◯ | | |
| 13 | 26695 | | | | | | | |
| 14 | HPK5 | | | ◯ | ◯ | | ◯ | ◯ |
| 15 | HPKT510 | ◯ | | | | ◯ | | |
| 16 | 11637 | ◯ | ◯ | | ◯ | | | |

TABLE 12
Total mineral concentration = 2,500 mg/kg

| No. | Bacteria name | A | B | C | D | E | F | G |
|---|---|---|---|---|---|---|---|---|
| 1 | KMT27 | ◯ | ◯ | ◯ | ◯ | ◯ | ◯ | ◯ |
| 2 | KMT31 | ◯ | ◯ | ◯ | ◯ | ◯ | | |
| 3 | KMT40 | | ◯ | ◯ | | ◯ | ◯ | |
| 4 | KMT44 | ◯ | ◯ | | ◯ | | ◯ | ◯ |
| 5 | KMT45 | ◯ | ◯ | ◯ | ◯ | ◯ | ◯ | ◯ |
| 6 | KMT46 | ◯ | ◯ | ◯ | ◯ | ◯ | ◯ | ◯ |
| 7 | KMT47 | ◯ | ◯ | ◯ | ◯ | ◯ | ◯ | ◯ |
| 8 | KMT50 | ◯ | ◯ | ◯ | ◯ | ◯ | | |
| 9 | NY1 | | | | | | ◯ | ◯ |
| 10 | NY8 | | | ◯ | ◯ | | | |
| 11 | NY11 | | | | | | | |
| 12 | NY31 | | ◯ | ◯ | ◯ | ◯ | | |
| 13 | 26695 | | | | | | | ◯ |
| 14 | HPK5 | | | ◯ | ◯ | | ◯ | ◯ |
| 15 | HPKT510 | ◯ | | | | ◯ | | |
| 16 | 11637 | ◯ | ◯ | | ◯ | | | |

TABLE 13
Total mineral concentration = 3,500 mg/kg

| No. | Bacteria name | A | B | C | D | E | F | G |
|---|---|---|---|---|---|---|---|---|
| 1 | KMT27 | ◯ | ◯ | ◯ | ◯ | ◯ | ◯ | ◯ |
| 2 | KMT31 | ◯ | ◯ | ◯ | ◯ | ◯ | ◯ | ◯ |
| 3 | KMT40 | ◯ | ◯ | ◯ | ◯ | ◯ | ◯ | ◯ |
| 4 | KMT44 | ◯ | ◯ | ◯ | ◯ | ◯ | ◯ | ◯ |
| 5 | KMT45 | ◯ | ◯ | ◯ | ◯ | ◯ | ◯ | ◯ |
| 6 | KMT46 | ◯ | ◯ | ◯ | ◯ | ◯ | ◯ | ◯ |
| 7 | KMT47 | ◯ | ◯ | ◯ | ◯ | ◯ | ◯ | ◯ |
| 8 | KMT50 | ◯ | ◯ | ◯ | ◯ | ◯ | ◯ | ◯ |
| 9 | NY1 | ◯ | ◯ | ◯ | ◯ | ◯ | ◯ | ◯ |
| 10 | NY8 | | | ◯ | ◯ | | | |
| 11 | NY11 | ◯ | | | | ◯ | ◯ | ◯ |
| 12 | NY31 | | ◯ | ◯ | ◯ | ◯ | | |
| 13 | 26695 | | | | | | | |
| 14 | HPK5 | | | ◯ | ◯ | | ◯ | ◯ |
| 15 | HPKT510 | ◯ | | | | ◯ | | |
| 16 | 11637 | ◯ | ◯ | | ◯ | | | |

TABLE 14
Total mineral concentration = 5,900 mg/kg

| No. | Bacteria name | A | B | C | D | E | F | G |
|---|---|---|---|---|---|---|---|---|
| 1 | KMT27 | ◯ | ◯ | ◯ | ◯ | ◯ | ◯ | ◯ |
| 2 | KMT31 | ◯ | ◯ | ◯ | ◯ | ◯ | ◯ | ◯ |
| 3 | KMT40 | ◯ | ◯ | ◯ | ◯ | ◯ | ◯ | ◯ |
| 4 | KMT44 | ◯ | ◯ | ◯ | ◯ | ◯ | ◯ | ◯ |
| 5 | KMT45 | ◯ | ◯ | ◯ | ◯ | ◯ | ◯ | ◯ |
| 6 | KMT46 | ◯ | ◯ | ◯ | ◯ | ◯ | ◯ | ◯ |
| 7 | KMT47 | ◯ | ◯ | ◯ | ◯ | ◯ | ◯ | ◯ |
| 8 | KMT50 | ◯ | ◯ | ◯ | ◯ | ◯ | ◯ | ◯ |
| 9 | NY1 | ◯ | ◯ | ◯ | ◯ | ◯ | ◯ | ◯ |
| 10 | NY8 | ◯ | ◯ | ◯ | ◯ | ◯ | ◯ | ◯ |
| 11 | NY11 | ◯ | ◯ | ◯ | ◯ | ◯ | ◯ | ◯ |
| 12 | NY31 | ◯ | ◯ | ◯ | ◯ | ◯ | ◯ | ◯ |
| 13 | 26695 | ◯ | ◯ | ◯ | ◯ | ◯ | ◯ | ◯ |
| 14 | HPK5 | ◯ | ◯ | ◯ | ◯ | ◯ | ◯ | ◯ |
| 15 | HPKT510 | ◯ | ◯ | ◯ | ◯ | ◯ | ◯ | ◯ |
| 16 | 11637 | ◯ | ◯ | ◯ | ◯ | ◯ | ◯ | ◯ |

TABLE 15
Total mineral concentration = 11,800 mg/kg

| No. | Bacteria name | A | B | C | D | E | F | G |
|---|---|---|---|---|---|---|---|---|
| 1 | KMT27 | ◯ | ◯ | ◯ | ◯ | ◯ | ◯ | ◯ |
| 2 | KMT31 | ◯ | ◯ | ◯ | ◯ | ◯ | ◯ | ◯ |
| 3 | KMT40 | ◯ | ◯ | ◯ | ◯ | ◯ | ◯ | ◯ |
| 4 | KMT44 | ◯ | ◯ | ◯ | ◯ | ◯ | ◯ | ◯ |
| 5 | KMT45 | ◯ | ◯ | ◯ | ◯ | ◯ | ◯ | ◯ |
| 6 | KMT46 | ◯ | ◯ | ◯ | ◯ | ◯ | ◯ | ◯ |
| 7 | KMT47 | ◯ | ◯ | ◯ | ◯ | ◯ | ◯ | ◯ |
| 8 | KMT50 | ◯ | ◯ | ◯ | ◯ | ◯ | ◯ | ◯ |
| 9 | NY1 | ◯ | ◯ | ◯ | ◯ | ◯ | ◯ | ◯ |
| 10 | NY8 | ◯ | ◯ | ◯ | ◯ | ◯ | ◯ | ◯ |
| 11 | NY11 | ◯ | ◯ | ◯ | ◯ | ◯ | ◯ | ◯ |
| 12 | NY31 | ◯ | ◯ | ◯ | ◯ | ◯ | ◯ | ◯ |
| 13 | 26695 | ◯ | ◯ | ◯ | ◯ | ◯ | ◯ | ◯ |
| 14 | HPK5 | ◯ | ◯ | ◯ | ◯ | ◯ | ◯ | ◯ |
| 15 | HPKT510 | ◯ | ◯ | ◯ | ◯ | ◯ | ◯ | ◯ |
| 16 | 11637 | ◯ | ◯ | ◯ | ◯ | ◯ | ◯ | ◯ |

Increasing the total mineral concentration in mineral-rich waters prepared from deep sea water significantly increased the types of bacterial strains whose proliferation could be inhibited, and once the total mineral concentration reached 5,900 mg/kg, these mineral-rich waters were found effective in inhibiting the proliferation of all types of Helicobacter pylori strains. In addition, the effects of inhibiting the proliferation of Helicobacter pylori strains became particularly prominent when the magnesium concentration was high.

1. A method for inhibiting the proliferation and migration of Helicobacter pylori strains using a substance containing minerals.

2. The method for inhibiting the proliferation and migration of Helicobacter pylori strains according to claim 1, characterized in that the mineral content per kilogram is 40 to 5,900 mg.

3. The method for inhibiting the proliferation and migration of Helicobacter pylori strains according to claim 1, characterized in that the substance containing minerals contains at least one or more of magnesium, calcium, sodium and potassium.

4. The method for inhibiting the proliferation and migration of Helicobacter pylori strains according to claim 1, characterized in that the substance containing minerals is a liquid.

5. The method for inhibiting the proliferation and migration of Helicobacter pylori strains according to claim 1, characterized in that the substance containing minerals is derived from deep sea water.

6. A beverage or food for inhibiting the proliferation and migration of Helicobacter pylori strains according to claim 1.

7. A method for inhibiting the proliferation and migration of Helicobacter pylori strains, comprising placing Helicobacter pylori strains in contact with a substance containing minerals in an amount effective to inhibit the proliferation and migration of Helicobacter pylori strains.

8. The method according to claim 7, wherein the substance contains the mineral in an amount of 40 to 5,900 mg per kilogram of the substance.

9. The method according to claim 7, wherein the minerals includes magnesium, calcium, sodium, and potassium wherein magnesium is contained more than calcium, sodium, and potassium.

10. The method according to claim 7, wherein the substance containing minerals is a liquid.

11. The method according to claim 7, wherein the substance containing minerals is deep sea water.

12. The method according to claim 7, wherein the Helicobacter pylori strains are strains inhabitating the duodenum and/or stomach.
